(19) United States
(12) Patent Application Publication
(10) Pub. No.: US 2024/0265527 A1
(43) Pub. Date: Aug. 8, 2024

(54) MEDICAL INFORMATION PROCESSING DEVICE, MEDICAL INFORMATION PROCESSING METHOD, AND NON-TRANSITORY COMPUTER READABLE MEDIUM

(71) Applicant: CANON MEDICAL SYSTEMS CORPORATION, Otawara-shi (JP)

(72) Inventor: Takahiko NISHIOKA, Otawara (JP)

(73) Assignee: CANON MEDICAL SYSTEMS CORPORATION, Otawara-shi (JP)

(21) Appl. No.: 18/427,906

(22) Filed: Jan. 31, 2024

(30) Foreign Application Priority Data
Feb. 2, 2023 (JP) 2023-014896
Jan. 30, 2024 (JP) 2024-012259

(51) Int. Cl. G06T 7/00; G06T 7/12; G06T 7/62; G06V 10/25; G16H 30/40

(52) U.S. Cl. CPC G06T 7/0012; G06T 7/12; G06T 7/62; G06V 10/25; G16H 30/40; G06T 2207/30008

(57) ABSTRACT

A medical information processing device includes a processor. The processor is configured to execute bone segmentation of an image and to acquire first data, execute, based on the first data, organ segmentation of the image, and acquire second data.

### CROSS REFERENCE TO THE RELATED APPLICATIONS

This application is based upon and claims the benefit of priority from the Japanese Patent Application No. 2023-014896, filed on Feb. 2, 2023, and the Japanese Patent Application No. 2024-012259, filed on Jan. 30, 2024, the entire contents of which are incorporated herein by reference.

### FIELD

Embodiments and drawings relate to a medical information processing device, a medical information processing method, and a non-transitory computer readable medium.

### BACKGROUND

With the progress of machine learning techniques including deep learning, a learned neural network has been widely used for various inferences. In the medical field as well, there is increasing demand for systemic organ segmentation. Conventionally, there has been a method of implementing segmentation of each organ by a plurality of segmentation engines.

Although an image of the whole body or an image of each part is input to these automatic processing servers, which organ is photographed is unknown only with part information. Accordingly, there is a problem in that multi-organ segmentation cannot be performed under an appropriate input condition.

### BRIEF DESCRIPTION OF DRAWINGS

FIG. 1 is a block diagram schematically illustrating an example of a medical information processing system according to an embodiment;

FIG. 2 is a flowchart illustrating an example of processing of a medical information processing device according to the embodiment;

FIG. 3 is a block diagram schematically illustrating an example of the medical information processing system according to the embodiment; and

FIG. 4 is a flowchart illustrating an example of processing of the medical information processing device according to the embodiment.

### DETAILED DESCRIPTION

According to one embodiment, a medical information processing device includes a processor. The processor is configured to execute bone segmentation of an image and to acquire first data, execute, based on the first data, organ segmentation of the image, and acquire second data.

Hereinafter, embodiments will be described with reference to the drawings. In the present disclosure, in a mode in which information processing by software is specifically implemented by using hardware resources, the software is executed by a program, and the software can be implemented by the program itself or a non-transitory computer readable medium storing the program. Furthermore, in the various data in the present specification and the drawings, data mainly used by an information processing device is typically digital data.

### First Embodiment

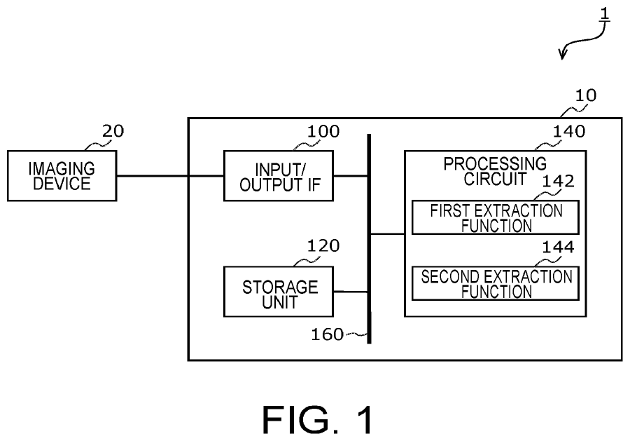

FIG. 1 is a diagram schematically illustrating an example of a medical information processing system 1 according to an embodiment. The medical information processing system 1 includes, for example, a medical information processing device 10 and an imaging device 20. For example, the medical information processing system 1 acquires data (second data) obtained by segmenting a region of an organ based on data (first data) obtained by bone segmentation in a computer tomography (CT) image of the whole body or a partial region of the body to be input.

The medical information processing device 10 acquires data obtained by segmenting organs from the CT image acquired by the imaging device 20. The generated data of organ segmentation can be used, for example, to extract a region of an organ from an input image captured by CT or to determine an organ in a region to be diagnosed. It is noted that other necessary configurations (not illustrated) for implementing the functions of the medical information processing device 10 may be included.

It is noted that, although illustrated as the medical information processing system 1, the medical information processing device 10 is not limited thereto, and may be provided as a part of a device (for example, the imaging device 20) that acquires a CT image, such as an X-ray CT device, as a non-limiting example.

The medical information processing device 10 includes an input/output interface (input/output I/F 100), a storage unit 120, and a processing circuit 140, which are connected to each other via a bus 160 or directly connected to each other as necessary.

The input/output I/F 100 is an interface that connects the inside and the outside of the medical information processing device 10. For example, the medical information processing device 10 acquires a CT image from the imaging device 20 via the input/output I/F 100. The medical information processing device 10 acquires, for example, data related to various bone segmentation or data related to various organ segmentation via the input/output I/F 100. The data related to bone segmentation and the data related to organ segmentation may be dictionary data, and can be referred to by the processing circuit 140 as a dictionary.

The storage unit 120 can store the data acquired via the input/output I/F 100. In addition, the storage unit 120 can store data used by the processing circuit 140. In a case where information processing by software is specifically implemented by using the processing circuit 140 which is a hardware resource for each function, a program, an execution file, and the like related to the information processing can be stored in the storage unit 120. In addition, data being processed in the processing circuit 140 may be temporarily stored in the storage unit 120, and data generated by the processing circuit 140 may be stored in the storage unit 120. The storage unit 120 may include one or a plurality of storage circuits.

The processing circuit 140 executes processing of generating and outputting the data related to organ segmentation in the medical information processing device 10. The processing circuit 140 has, for example, a first extraction function 142 and a second extraction function 144.

The first extraction function 142 executes bone segmentation on data (for example, the CT image) acquired from the imaging device 20, an external storage other than the imaging device 20, or the like. The first extraction function 142 generates first data which is information on the bone in the bone segmented CT image.

In the present disclosure, the bone segmentation means, for example, that information on a position, a shape, and the like of a bone in the CT image and information for identifying a bone, such as a type and an identifier of the bone, are segmented in association with each other. The medical information processing device 10 can include the first extraction function 142 as a first extraction unit.

The second extraction function 144 executes the organ segmentation of the CT image based on the first data generated by the first extraction function, and generates second data which is information on an organ in the CT image. The organ segmentation means, for example, that information on a position, a shape, and the like of an organ in the CT image and information for identifying the organ, such as a type and an identifier of the organ, are segmented in association with each other. The medical information processing device 10 can include the second extraction function 144 as a second extraction unit.

The second extraction function 144 acquires, for example, information on organs existing around the bone from the dictionary based on information on the bone segmented in the first data. For example, the second extraction function 144 can determine a type of the bone from the first data and search for a peripheral organ according to the type of the bone. The second extraction function 144 executes organ segmentation based on the acquired information on the organ.

The second extraction function 144 can further acquire information on the organs existing around the bone from the dictionary with reference to the size of the bone segmented in the first data.

This dictionary is, for example, a dictionary for converting data into organ segmented information when the data related to a region in the CT image is input. More specifically, for example, when a region in the CT image is designated, the dictionary may be a dictionary for acquiring mask information or domain information indicating the same organ region as the region.

By referring to the size of the bone in conjunction with the type of the bone, the second extraction function 144 can improve accuracy of organ segmentation even for subjects having greatly different sizes, such as adults and children.

The second extraction function 144 sets, for example, a first region of interest based on, for example, information indicating a region of an organ that can be specified from the position and the size of the bone from the type of the bone and the size of the bone acquired in the bone segmentation. Then, the second extraction function 144 inputs data of the first region of interest to the dictionary, thereby extracting a region indicating the organ. The first region of interest is set as a region having a size determined from the type of the bone and the size of the bone. The region of interest may be data represented by a volume of interest (VOI). Simply, the second extraction function 144 can set VOI of a cube in which a length of one side is designated as the first region of interest. The cube is shown as an example, and the present invention is not limited thereto, and it is also possible to designate VOI of a rectangular parallelepiped, a sphere, a spheroid, or any shape or size.

As a specific example, the second extraction function 144 specifies an organ existing near the sternum as a heart, and sets the first region of interest indicating the heart based on the position of the sternum and the size of the sternum. The second extraction function 144 extracts a region obtained by converting the first region of interest with the dictionary as a region of the heart, and stores the region in the storage unit 120 as organ segmentation data (second data).

The second extraction function 144 uses, for example, a plurality of first regions of interest relatively indicating the region of the heart with respect to the sternum instead of one first region of interest, and convert the plurality of first regions of interest with the dictionary, thereby making it possible to obtain a region from which the heart in the organ segmentation data is extracted.

As another example, the second extraction function 144 can acquire the segmentation region of the liver via the dictionary by setting the first region of interest, which is the liver with respect to the position and the size of the rib 10, and can acquire the segmentation region of the psoas major muscle via the dictionary by setting the first region of interest, which is the psoas major muscle with respect to the position and the size of the pelvis.

The second extraction function 144 can cut out not only organs but also muscles, main blood vessels, and other anatomically meaningful regions as segmentation. The second extraction function 144 may include a plurality of dictionaries according to a plurality of types of segmentation, such as a dictionary for conversion of segmenting an organ and a dictionary for conversion of segmenting a muscle. In this manner, by designating a bone and an organ or the like near the bone, various segmentation can be acquired. In addition, the second extraction function 144 can use a plurality of different dictionaries to acquire segmentation of the same organ.

In addition, the second extraction function 144 can change the size of the first region of interest according to the organ segmentation to which the data acquired from the dictionary is input. The second extraction function 144 can determine the size of the first region of interest according to, for example, the size of the grid in the organ segmentation, granularity at which data can be input, and density.

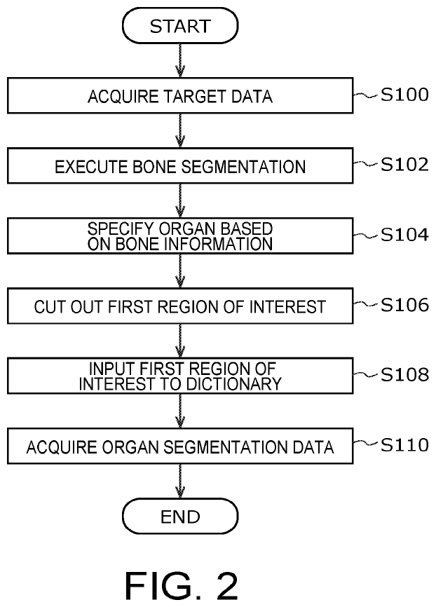

FIG. 2 is a flowchart illustrating an example of processing of the medical information processing device 10 according to the embodiment.

The medical information processing device 10 acquires data to be segmented via the input/output I/F 100 (S100). This data may be, for example, image data, model data, or the like acquired from various CT devices. The acquired data may be stored in the storage unit 120.

The first extraction function 142 of the processing circuit 140 executes bone segmentation of the target data (S102). The bone segmentation can be performed using any technique. For example, the processing circuit 140 executes the bone segmentation based on a learned model or a rule-based model for converting the image data included in the storage unit 120 into bone segmentation data.

The second extraction function 144 of the processing circuit 140 specifies an organ existing near a bone based on data of a bone type acquired by the bone segmentation (S104). For example, the second extraction function 144 designates information on a region in which an organ desired to be known exists as a first region of interest, and acquires which organ is the first region of interest from a type of bone and a relative position between the bone and the region. It is noted that the second extraction function 144 can also acquire, from the bone segmentation, information on a bone located closest to the region in which an organ desired to be known exists, and specify the organ based on the information on the bone.

The second extraction function 144 cuts out the first region of interest including at least the above region or including at least a region having the same domain as that of the above region based on the size of the bone (S106). For example, the second extraction function 144 may acquire information on the first region of interest having a size relative to the size of the bone. According to this processing, the second extraction function 144 can acquire the information on the first region of interest regardless of the physique.

The second extraction function 144 inputs the cut-out first region of interest to a dictionary and acquires information on the organ (S108). For example, the second extraction function 144 prepares a dictionary reflecting the size of the first region of interest, and inputs the first region of interest to the dictionary, thereby acquiring information such as a domain and a mask of the first region of interest.

At a timing of inputting the first region of interest to the dictionary, the second extraction function 144 may input the first region of interest to the dictionary after enlarging or reducing the size of the first region of interest and adjusting the position thereof so that the size information on the bone matches the size information on the bone in the dictionary. By using the dictionary in this manner, the second extraction function 144 can execute appropriate region extraction in consideration of the skeleton. It is noted that the second extraction function 144 may convert not the data to be processed but the information on the dictionary into the size of the acquired bone and extract the region.

The second extraction function 144 acquires organ segmentation data on the assumption that the extracted region is the region of the organ specified in S104 (S110).

It is noted that, as described above, a target for region extraction is not limited to an organ. When the target for region extraction is not an organ, for example, the processing circuit 140 can appropriately segment the region to be extracted in S110 by appropriately changing the target specified in S104 and the dictionary used in S108.

The medical information processing device 10 can output the second data generated by the second extraction function 144 to the outside via the input/output I/F 100 or can store the second data in the storage unit 120. A medical practitioner can acquire the information on the region of interest in the CT image in more detail by referring to the second data.

In FIG. 2, an example of extracting a region such as an organ related to a region of interest has been described, but the medical information processing device 10 according to the present embodiment is not limited to this processing. For example, the medical information processing device 10 can perform segmentation including a plurality of main organs by setting the first region of interest from region information on a main organ and repeating segmentation of the organ based on information on the bone after bone segmentation. According to this processing, the medical information processing device 10 can further execute segmentation of organs and the like at any particle size without being limited to the main organ.

As described above, with the medical information processing device 10 of the present embodiment, it is possible to generate organ segmentation based on the type and the size of the bone. This organ segmentation can be generated based on the information on bone segmentation that is more robust than general organ segmentation.

In order to generate the general organ segmentation, adult data is often used, and it is difficult to generate accurate organ segmentation of a child because of a small amount of child data. On the other hand, according to the method of the present disclosure, appropriate extraction can be performed using a learned model trained using adult data, thereby making it possible to generate organ segmentation information based on more robust bone segmentation that has a small difference between an adult and a child and between individuals.

Therefore, with the medical information processing device 10, it is possible to provide highly accurate organ segmentation for patients of various physiques.

### Second Embodiment

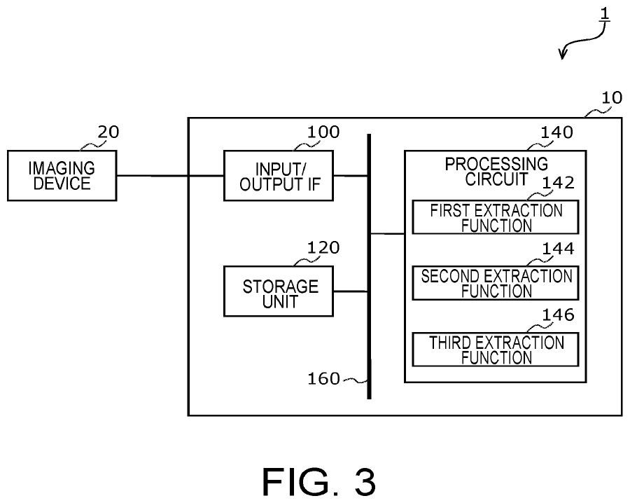

FIG. 3 is a block diagram schematically illustrating an example of the medical information processing system 1 according to the embodiment. The medical information processing device 10 may include a third extraction function 146 in the processing circuit 140 in addition to the configuration of the first embodiment described above.

The third extraction function 146 is a function of further extracting information from the segmentation information on the organ extracted by the second extraction function 144. The third extraction function 146 can be provided as a third extraction unit in the medical information processing device 10.

The third extraction function 146 sets a second region of interest for the segmentation of the organ after the second extraction function acquires the segmentation information on the organ. The second region of interest may be a region included in the segmentation of an organ, or may be a region included in segmentation different from the organ.

For example, the third extraction function 146 sets the second region of interest outside the segmentation with respect to the acquired information on the organ. The third extraction function 146 can set, as the second region of interest, a region indicating another organ (another organ whose positional relationship can be grasped) whose positional relationship with the acquired organ is clear. The third extraction function 146 may set the position and the size of the second region of interest with respect to the acquired organ based on the type and the size of the bone used at a timing of setting the first region of interest.

The third extraction function 146 can extract, for example, the region of another organ by inputting the second region of interest to a dictionary.

In the medical information processing device 10, for example, the second extraction function 144 can acquire a region of a main organ, and the third extraction function 146 can extract a region of another organ (may be a muscle, a blood vessel, or the like) near the main organ.

In the above description, the extraction target is another organ, but the extraction target is not limited thereto. For example, the third extraction function 146 can set the second region of interest in which the positional relationship can be grasped in the region of the organ acquired based on the first region of interest. By setting this organ, the third extraction function 146 can execute segmentation for, for example, a blood vessel in the organ, a connection point with another organ, or any region in the organ.

Similarly to the above-described embodiment, the third extraction function 146 can change the dictionary to be used according to the object to be extracted.

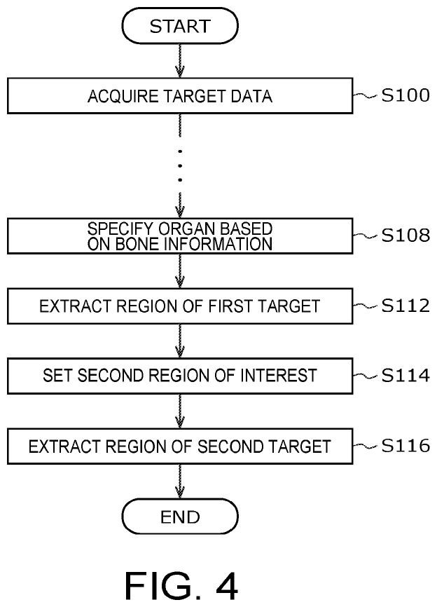

FIG. 4 is a flowchart illustrating processing of the medical information processing device 10 according to the present embodiment.

The processing procedures up to S108 are the same as those in the first embodiment described above.

After the processing in S108, the second extraction function 144 extracts a region of a first target with respect to the first region of interest (S110). As a non-limiting example, the first target may be a liver, and the second extraction function 144 cuts out the first region of interest of the liver from a rib 10 to extract region information on the first target.

The third extraction function 146 sets the second region of interest based on the region of the first target (S114). At this time, as described above, the third extraction function 146 may reflect, in the second region of interest, information such as the size of the bone extracted by the first extraction function 142. As a non-limiting example, the third extraction function 146 can set the second region of interest using a target such as the hepatic artery in the liver as a second target, or can set the second region of interest using a target outside the liver, such as the gallbladder, as the second target.

The third extraction function 146 inputs the second region of interest to the dictionary to extract a region of the second target (S116).

As described above, with the medical information processing system 1 of the present embodiment, the third extraction function 146 can further execute segmentation on another organ or the like based on the information on segmentation of the organ or the like extracted from the segmentation of the bone by the second extraction function 144.

Since the organ or the like extracted by the second extraction function 144 is acquired from information that is robust to a difference in physique or the like, information on the organ or the like whose position or the like can be grasped can also be acquired as information that is robust to a difference in physique or the like by the third extraction function 146.

The input/output I/F 100 may include a display (display unit) that displays a calculation result (extraction result) in the medical information processing device 10. Based on a display content on the display, a user can also make a request for a change in display, recalculation, or the like via the input/output I/F 100 that operates as an input unit. In this manner, the medical information processing device 10 can execute appropriate processing based on the request from the user. Furthermore, the medical information processing device 10 can also execute calculation by interactive exchange with the user based on an output content.

Hereinafter, reception of a request from the user via the input/output I/F 100 and calculation control of the medical information processing device 10 for the reception will be described with some non-limiting specific examples.

The medical information processing device 10 may acquire a candidate region of an organ to be specified by the second extraction function 144 after the first extraction function 142 executes the bone segmentation. The medical information processing device 10 may display the candidate region extracted by the second extraction function 144 via the input/output I/F 100. The user can view the displayed region and request whether to execute the organ segmentation based on the region.

The medical information processing device 10 can present the candidate region on the display unit so that the candidate region is indicated by a frame of any shape such as a rectangular frame, and the user is instructed whether to execute the organ segmentation on the region indicated by this frame. The medical information processing device 10 can execute the organ segmentation in the region by displaying a button for execution on the display unit and by allowing the user to click or tap the button. The second extraction function 144 can continuously execute the organ segmentation according to the request from the user acquired via the input unit.

Alternatively, a plurality of candidate regions may be displayed, and the user may select any one of the candidate regions via the input unit and perform the organ segmentation.

As described above, the medical information processing device 10 can define an organ to be a target in accordance with a request from the user after segmentation of a bone and specification of a reference bone.

The medical information processing device 10 can also acquire bone information serving as a reference for specifying an organ from the user via the input unit. The medical information processing device 10 can acquire, for example, information indicating that the rib 5 to 10 is a reference bone of the liver from the user via the input unit as a character or a piece of data. Furthermore, as another example, the medical information processing device 10 can display the pelvis on the display unit, and can set at which position of the pelvis a box is to be displayed by allowing the user to designate the box at any position via the input unit.

In this manner, the medical information processing device 10 can define a bone serving as a reference of a target organ by an explicit request from the user.

For example, the medical information processing device 10 may 10 may infer the sex, age, and the like of a target on which segmentation is to be executed from bone information. The medical information processing device 10 can further change the position and the size of the displayed box based on an inference result of the sex, the age, and the like.

For example, in the case of an elderly person, there is a case in which a spine is bent. In such a case, the medical information processing device 10 can estimate a compression state of an organ based on a bending degree of a bone if the bone is used as a landmark. Furthermore, in a case where it is estimated that the organ is compressed, the medical information processing device 10 can output a message indicating that the position of the organ may be changed via the display unit (or another output I/F).

The medical information processing device 10 can also output a warning based on these estimation results. This warning can also be pop-up displayed so as to overlap a result of the organ segmentation on the display unit or in a display region different from the result of the organ segmentation. Furthermore, the medical information processing device 10 can output a warning by making some notification to the display unit or some notification to the output I/F other than the display unit.

By performing such an output, it is possible to perform organ segmentation and detect and output an abnormality of the body in a target patient or the like. For this inference result, the medical information processing device 10 can output the segmented organ in an easy-to-understand manner by, for example, changing a color of the segmented organ or blinking the segmented organ.

In this manner, the medical information processing device 10 can detect an abnormality or the like (including a position at which the abnormality is estimated to have occurred) based on a specific result as a result of the organ segmentation, and output a notification or the like to the user.

In the above specific example, the processing based on the result of organ segmentation processed by the second extraction function 144 has been described, but the present invention is not limited thereto, and the above specific example can also be applied based on the result extracted by the third extraction function 146. That is, each processing described above may be processing executed by at least one circuit included in at least the processing circuit 140.

In the above embodiment, the input interface can be implemented by a trackball for performing various settings, a switch button, a mouse, a keyboard, a touch pad for performing an input operation by touching an operation surface, a touch screen in which a display screen and the touch pad are formed to be integrated with each other, a non-contact input circuit using an optical sensor, a sound input circuit, and the like. The input interface is connected to a control circuit, converts an input operation received from an operator into an electrical signal, and outputs the electrical signal to the control circuit. It is noted that, in the present specification, the input interface is not limited to one including physical operation components such as a mouse and a keyboard. For example, examples of the input interface include an electrical signal processing circuit that receives an electrical signal corresponding to an input operation from an external input device provided separately from the device and outputs the electrical signal to the control circuit.

Further, in the above embodiment, each processing function of the information processing function is recorded in a storage circuit in the form of a program executable by a computer. The processing circuit can include a processor. For example, the processing circuit reads a program from the storage circuit and executes the program to implement a function corresponding to each program. In other words, the processing circuit in a state of reading each program has each function illustrated in the processing circuit illustrated in the drawings. It is noted that, in the drawings, it has been described that each processing function is implemented by a single processor. However, a processing circuit may be configured by combining a plurality of independent processors, and the functions thereof may be implemented by each processor executing a program. Furthermore, in the drawings, it has been described that a single storage circuit stores a program corresponding to each processing function, but a plurality of storage circuits may be arranged in a distributed manner, and the processing circuit may read the corresponding program from each storage circuit.

In the above description, an example has been described in which the “processor” reads a program corresponding to each function from the storage circuit and executes the program, but the embodiment is not limited thereto. The term “processor” can mean, for example, a circuit such as a central processing unit (CPU), a graphics processing unit (GPU), an application specific integrated circuit (ASIC), or a programmable logic device (for example, a simple programmable logic device (SPLD), a complex programmable logic device (CPLD), and a field programmable gate array (FPGA)). In a case where the processor is, for example, the CPU, the processor implements a function by reading and executing a program stored in the storage circuit. On the other hand, when the processor is the ASIC, instead of storing the program in the storage circuit, the function is directly incorporated as a logic circuit in a circuit of the processor. It is noted that each processor of the present embodiment is not limited to being configured as a single circuit for each processor, and a plurality of independent circuits may be combined to be configured as one processor to implement the function. Further, a plurality of components in the drawings may be integrated into one processor to implement the function.

According to at least one embodiment described above, robust organ segmentation can be provided.

Although several embodiments have been described, these embodiments have been presented as examples, and are not intended to limit the scope of the invention. These embodiments can be implemented in various other forms, and various omissions, substitutions, changes, and combinations of the embodiments can be made without departing from the gist of the invention. These embodiments and modifications thereof are included in the scope and gist of the invention and are included in the invention described in the claims and the scope equivalent thereto.

1. A medical information processing device comprising a processor configured to:
execute bone segmentation of an image and to acquire first data; and
execute, based on the first data, organ segmentation of the image and to acquire second data.

2. The medical information processing device according to claim 1, wherein
the processor is configured to acquire, from information on a segmented bone in the first data, information on an organ existing around the bone based on a type of the bone, and to acquire information on the segmented organ as the second data.

3. The medical information processing device according to claim 2, wherein
the processor is configured to set a first region of interest based on a size of the bone in the first data, to acquire the information on the organ in the first region of interest based on a dictionary, and to generate the second data.

4. The medical information processing device according to claim 3, wherein
the processor is configured to select a dictionary from the plurality of dictionaries according to a type of the organ, to set a size of the first region of interest based on the selected dictionary, and to acquire information on segmentation from the information on the bone included in the first data using the selected dictionary.

5. The medical information processing device according to claim 4, wherein
the processor is further configured to set a second region of interest for the information on the organ acquired based on the first region of interest, and to further acquire information on another organ.

6. The medical information processing device according to claim 5, wherein
the processor is configured to set the second region of interest as a region of interest related to the other organ whose positional relationship with the organ is graspable.

7. The medical information processing device according to claim 4, wherein
the processor is further configured to set a second region of interest for the information on the organ acquired based on the first region of interest, and to further acquire information on an inside of the organ.

8. The medical information processing device according to claim 1, further comprising
a display configured to display and output information, wherein the display is configured to display:
information on a segmented bone in the first data acquired by the processor; and
a candidate region of an organ acquired by the second extraction unit based on the first data.

9. The medical information processing device according to claim 8, wherein
the processor is further configured to receive a request from a user, the processor receives the request from the user with respect to the candidate region of the organ displayed on the display unit, and executes segmentation of the organ based on the request received by the input unit.

10. The medical information processing device according to claim 1, wherein
the processor is further configured to estimate sex or age based on a result of the bone segmentation, to determine whether an abnormality has occurred based on a result of the organ segmentation, and to specify and output a position of the abnormality when the abnormality has occurred.

11. A medical information processing method comprising:
executing, by a processor, bone segmentation of an image to acquire first data; and
executing, by the processor, organ segmentation of the image based on the first data to acquire second data.

12. A non-transitory computer readable medium having a program stored therein and configured to cause a processing circuit to execute:
bone segmentation of an image and acquiring first data; and
organ segmentation of the image based on the first data and acquiring second data.
